(19) United States
(12) Patent Application Publication
(10) Pub. No.: US 2005/0106232 A1
(43) Pub. Date: May 19, 2005

(54) Biocompatible composite capsules

(75) Inventors: Yves Frere, Holtzheim (FR); Louis Danicher, Strabourg (FR)

Correspondence Address: YOUNG & THOMPSON, 745 SOUTH 23RD STREET, 2ND FLOOR, ARLINGTON, VA, 22202, US

(73) Assignee: Centre National De La Recherche Scientifique (C.N.R.S.), Paris (FR)

(21) Appl. No.: 10/507,606

(22) PCT Filed: Mar. 27, 2003
(86) PCT No.: PCT/FR03/00975

(30) Foreign Application Priority Data
Mar. 28, 2002 (FR) 02/03939

(51) Int. Cl. A61K 9/48; B01J 13/04

(52) U.S. Cl. 424/451; 264/4.1

(57) ABSTRACT

The invention relates to a method of preparing composite capsules by means of interfacial polycondensation in a dispersed medium of at least two different monomers. The invention is characterised in that the aforementioned polycondensation is performed in a gas in the liquid and/or supercritical state. The composite capsules thus obtained can be used in the pharmaceutical, veterinary, phytosanitary or cosmetic fields for the delayed, controlled, prolonged and/or controlled release and/or the vectorisation of active principle(s) contained therein.

The present invention relates to biocompatible composite capsules, to their process of preparation and to their uses, in particular in the therapeutic, biomedical, pharmaceutical, veterinary, paramedical, plant-protection and cosmetic fields.

In a great number of industrial fields, such as, for example, the pharmaceutical industry (both therapeutic and veterinary) or the cosmetics industry, but also for plant-protection products (for example, agrochemical and detergent products) and printing industry products (inks), to mention only some of them, it is increasingly desired to be able to isolate an active substance from the medium which surrounds it (reference is then made to retention of the active material) in order to promote better preservation and/or to promote delayed, sustained or prolonged release or also to provide vectorization of said active substance.

The techniques for retaining active substances are methods which consist in preparing separate particles composed of a material which coats said substance. These particles exhibit sizes of between a few nanometers and a few millimeters. These include reservoir particles composed of a continuous solid membrane which encases a core filled with active material. These reservoir particles are known as capsules.

The term “encapsulated product” indicates that a product, denoted generally by “active substance”, that is to say the therapeutic, veterinary or agrochemical active principle, the cosmetic product or the detergent product or alternatively the printing ink, is enclosed, in the solid or liquid state, indeed even gas state, alone or in combination with formulating agents, in a hollow body, the capsule, in order to isolate it from the external medium.

The capsule is thus generally composed of a membrane, the role of which is, first, to isolate the active substance from the external medium and, secondly, to make possible better preservation of said active substance, to it make possible a vectorization and/or an immediate, prolonged, delayed and/or controlled release of this encapsulated active substance.

Numerous encapsulation techniques are already known and in particular the synthesis of capsules by coating or coacervation, which technique involves the use of a polymer as coating agent with the solubility limits specific to the latter (cf. U.S. Pat. No. 2,800,457).

U.S. Pat. No. 6,183,783, for example, discloses a process for the preparation of microcapsules comprising an active substance which is coated with a substantially polar polymer film. The process for the formation of the capsule uses the conventional method of coacervation in organic solvent medium. This organic solvent is subsequently removed by several extraction cycles with supercritical carbon dioxide. There are many disadvantages to this process, in particular that of carrying out numerous extractions of solvent and of the need to recycle the latter. In addition, the capsules resulting from this coacervation technique comprise a membrane in the form of a continuous polymer film which isolates the active substance by a barrier effect, which slows down the deterioration in the coating layer and consequently the diffusion of the active substance.

Other techniques involve emulsion polymerization, generally of latex particles with a diameter of less than a micron. The disadvantage of this method is mainly the fact that the structure of the particles obtained is difficult to control, indeed even uncontrollable.

Yet other techniques, such as the synthesis of solid particles, the synthesis of vesicles or alternatively the synthesis of capsules by the fluidized bed process, by coating or by spraying are also employed industrially.

Finally, another technique involves the technique of interfacial polycondensation in a dispersed medium, a technique disclosed, for example, by P. W. Morgan et al., J. Polym. Sci., 40, (1959), 299-327, for the synthesis of flat films. This technique has been adapted to the synthesis of minicapsules (R. Arshady, J. Microencap., 6(1), (1989), 1-10 and 13-28).

All these techniques, and in particular those which give better results, such as, for example, interfacial polycondensation, generally use, during the synthesis of said capsules, organic solvents. In point of fact, for applications in human or veterinary therapeutics, in the plant-protection field or in cosmetics, such organic solvents may prove to be insufficiently biocompatible and even, in some cases, toxic.

In addition to organic solvents, the encapsulation techniques employed very often prescribe the nature of the constituent polymer of the membrane, which polymer very often proves to possess little or no biocompatibility.

Furthermore, for example for uses in the medical and paramedical fields, it is essential to have available capsules which are all substantially of the same size or which at the very least exhibit a substantially identical encapsulation volume for all the capsules. This is because it is preferable, in such applications, for the dose of active substance encapsulated to be as constant as possible for a given volume of capsules.

Finally, and in addition to the biocompatibility of the capsules, the membrane must make it possible the release of the encapsulated substance, this release possibly resulting either from the rupture, that is to say from the physical and/or chemical and/or biological destruction, of the membrane or from the permeability of the membrane to said active substance.

Thus, the disadvantages related to the low biocompatibility, indeed even to the lack of compatibility, or to the toxicity, in combination or not with the difficulty of controlling the structure of the capsules and with the intrinsic properties of the membranes, means that only a few products encapsulated by interfacial polycondensation in a dispersed medium are currently commercially available in the medical and paramedical field.

However, there exist active substances highly effective for a very large number of uses, in particular in the medical and paramedical fields, the effectiveness of which can only be precisely and effectively displayed and/or controlled when the active principle is suitably vectorized, delivered, released, in a delayed, prolonged and/or controlled way, that is to say when the active substance is encapsulated.

In particular, for these applications in human or animal therapeutics or in the plant-protection and cosmetic fields, there exists an urgent and significant need to provide novel methods of encapsulation which can eliminate the disadvantages related to the methods of encapsulation of the prior art as defined above.

A first objective of the invention consists in providing a process for the preparation of biocompatible capsules, that is to say capsules which are compatible with and nontoxic to the living environment and in particular to human beings, animals and plants.

Another objective of the invention is to provide a process for the preparation of biocompatible capsules which makes it possible to control the structure of the membrane and the characteristics of said capsules, in particular the size.

According to another objective, the process for the preparation of biocompatible capsules according to the invention must make it possible to obtain capsules exhibiting a mechanical strength suited to the uses for which they are intended.

Another objective of the present invention is a process for the preparation of biocompatible capsules involving a constituent polymer of the membrane, the physicochemical properties of which making possible a delayed, prolonged and/or controlled release of the encapsulated active substance.

Yet other objectives, in addition to those set out above, will become apparent in the following description of the invention.

Thus, according to a first aspect, the present invention relates to a process for the preparation of composite capsules by interfacial polycondensation in a dispersed medium of at least two different monomers M₁ and M₂, characterized in that said polycondensation is carried out in a gas in the liquid and/or supercritical state.

The preparation process selected for the synthesis of the biocompatible capsules of the present invention is the interfacial polycondensation technique. This method makes possible optimum control of the size of the capsules but also of the degree of progression of the polymerization. In addition, this method offers a flexibility in the choice of the constituents of the membrane, said flexibility being much greater than with, for example, the coacervation technique.

Specifically, the polycondensation process at the interface of two liquids consists, generally, in creating a dispersion of one liquid in another. The two liquids have to be substantially immiscible with one another, such as a lipophobic liquid and a lipophilic liquid, for example an aqueous phase and an organic phase, thus forming a continuous phase (or dispersing phase) and a dispersed phase.

The term “direct system” will be used when the organic phase is dispersed in the aqueous phase (oil-in-water type dispersion) and the term “reverse system” will be used when the aqueous phase is dispersed in the organic phase (water-in-oil type dispersion).

The choice between direct system and reverse system is mainly guided by the nature of the active substance which it is desired to encapsulate. Thus, for fat-soluble active principles, it is preferable advantageously to prepare a dispersion in a direct system. On the other hand, for water-soluble active principles, the dispersion in a reverse system is favorable. Of course, the use of one or other of the systems can vary according to the intrinsic nature of the active principles to be encapsulated.

The membranes of the capsules are thus formed by polycondensation of at least two monomers, one of which is soluble in the dispersing phase and another of which is soluble in the dispersed phase.

Thus, to form the capsules, a solution comprising a monomer M₁ dissolved in a solvent S₁ is dispersed in a solution comprising a solvent S₂ not comprising reactive entities. Once the solution is dispersed and stabilized, the monomer M₂, soluble in S₂, of the dispersing phase is added. The condensation reaction between M₁ and M₂, which is optionally catalyzed, takes place at the interface of the dispersing phase and of the dispersed phase. The term “interfacial polycondensation” is then used. The ratio by volume of dispersing phase to dispersed phase is generally between 1/10 and 10/1.

More specifically, the process of the present invention comprises the following steps:
a) a solution of at least one monomer M₁ in a solvent S₁ comprising the substance to be encapsulated is added, with stirring, to a solution comprising a solvent S₂, in order to obtain a stabilized dispersion, S₁ or S₂ being a gas in the liquid and/or supercritical state;
b) a solution comprising at least one monomer M₂ present in the minimum amount of S₂ is added to this dispersion;
c) the polymerization is carried out by polycondensation at the interface of the two liquids;
d) the gas present in the reaction medium is removed; and
e) the capsules are extracted from the reaction medium.

As previously said, the principle of the interfacial polycondensation consists in preparing a dispersion of one solution in another, the two solutions being immiscible with one another. Thus, S₁ is a solvent for M₁ but is insoluble in S₂, which is a solvent for M₂. If S₁ is a solvent of the lipophobic phase, there will be present a dispersion of S₁ in S₂, that is to say of water-in-oil type (reverse system). Conversely, if S₁ is the solvent of the lipophilic phase, there will be present a dispersion of oil-in-water type (direct system).

The preferred process of the present invention is of reverse system type and, in this case, the solvent S₁ is advantageously water and the solvent S₂ is the gas in the liquid and/or supercritical state. Preferably, the gas is placed under temperature or pressure conditions such that it is in the liquid state throughout the duration of the synthesis.

An alternative consists in preparing the dispersion under temperature and pressure conditions where the gas is in the liquid state and then modifying these conditions in order to carry out the polycondensation reaction in the gas in the supercritical state.

In the process of the present invention, it is recommended for the dispersion obtained in stage a) to be stable, that is to say for the reaction medium not to undergo demixing, separation, settling, and the like. To this end, it is advantageous to use a surface-active agent, which can be of any type, ionic or nonionic, and which possesses the characteristic of being spontaneously adsorbed at the interface of the two liquids.

Such molecules, adsorbed at the interface, form a steric and/or electrostatic barrier which thus prevents the droplets and then the capsules in the course of formation from coalescing. For this reason, the surface-active agent which stabilizes the dispersion prevents the capsules from agglomerating with one another once the phase of growth of the membrane has ended.

The size of the capsules thus obtained is directly related to the size of the droplets present in the dispersion. The size of the latter is itself influenced by numerous parameters, the main ones of which are the speed and the duration of the dispersing, the nature and the concentration of the surfactant used, the nature, the viscosity and the volumic ratio of each of the dispersing and dispersed phases and, finally, the temperature at which the dispersing and the polycondensation reaction are carried out.

By virtue of the control of the various parameters described above, it is thus possible to obtain particles with a substantially uniform size. The size of the capsules can consequently be controlled so that the mean diameter is substantially constant, with a relatively low dispersity.

Thus, and according to the operating conditions defined above, the process of the present invention makes possible the preparation of capsules with sizes of between a few nanometers and a few millimeters. In the present invention, the term “nanocapsules” will be used when their mean external diameter is between approximately 0.01 μm and approximately 0.9 μm, the term “microcapsules” will be used when the mean diameter is between approximately 1 μm and approximately 50 μm, the term “minicapsules” will be used for a mean diameter of between 50 μm and approximately 500 μm and the term “millicapsules” will be used for mean diameter values of between approximately 0.5 mm and approximately 5 mm.

The process according to the present invention is particularly suitable for the synthesis of nanocapsules, microcapsules and minicapsules, that is to say for the preparation of capsules having a mean external diameter of between approximately 0.01 μm and approximately 500 μm, preferably between approximately 0.05 μm and approximately 300 μm. For uses in the medical and paramedical field, use will advantageously be made of nanocapsules, microcapsules and minicapsules having in particular a mean external diameter of between approximately 0.01 μm and approximately 500 μm, preferably between approximately 0.05 μm and approximately 300 μm.

The inventors have now discovered and developed a process for the preparation of biocompatible composite capsules by interfacial polycondensation in the dispersed medium, in which the solvent of the organic phase is a gas in the liquid or supercritical state or else in the liquid and then supercritical state.

It should be specified here that the term “biocompatible” describes entities which, when they are introduced or penetrate inside a living organism, do not produce negative effects on the latter and are subsequently eliminated, metabolized and/or assimilated by said organism. The term “biocompatible” also includes the entities for which the decomposition products within the living organism, such as the metabolites, are also biocompatible.

Generally, the interfacial polycondensation reactions in the dispersed medium require the use of two solvents which are substantially immiscible with one another. Water, which generally does not exhibit toxicity, is usually used as solvent of the lipophobic phase. Raw water can be used but water which is distilled, treated with ion-exchange media, sterilized or deionized by passing through an ion-exchange resin will be preferred, however.

On the other hand, the solvent of the organic phase, generally selected from conventional solvents, such as toluene, cyclohexane, carbon tetrachloride, chloroform, and the like, generally exhibits a more or less pronounced toxicity which is totally unacceptable for medical or paramedical uses.

It has therefore been necessary to find an organic solvent which can have the required properties necessary for being used in interfacial poly-condensation reactions in a dispersed medium and which is nontoxic to the living environment, that is to say which is biocompatible.

A particularly suitable solution consists in carrying out the interfacial polycondensation reaction using an organic solvent which has the solvating properties required for such a reaction and which can be easily removed at the end of the reaction. One type of solvent which is entirely suitable is a gaseous solvent which can be brought to the liquid and/or supercritical state during the interfacial polycondensation reaction. After the reaction and after returning to standard temperature and pressure conditions, said solvent is thus removed in the gas form.

Thus, suitable solvents for the interfacial polycondensation reaction according to the present invention comprise all gaseous compounds which have the solvating properties required for such a reaction, which are inert with respect to the strictly speaking interfacial polycondensation reaction and which exist in the liquid or supercritical form under specific temperature and/or pressure conditions.

Such gaseous compounds are, for example, air, oxygen, nitrogen, nitrous oxide, carbon dioxide, the rare gases or halogenated or nonhalogenated hydrocarbons, for example propane, butane, fluorocarbon compounds and others. As indicated above, these compounds are gases under standard temperature and pressure conditions. Mixtures of these compounds in all proportions can also be used. A very particularly preferred gaseous compound is carbon dioxide.

Furthermore, and from the viewpoint of the use of the biocompatible capsules according to the present invention in the therapeutic or veterinary fields and generally in any field relating to human, animal or plant health, use will advantageously be made of nontoxic gaseous compounds, that is to say compounds which are nontoxic to human beings, animals and/or plants.

Specifically, carbon dioxide is an organic solvent which is very little used as such but which exhibits, however, the desired advantages. This is because carbon dioxide is more or less inert with respect to the reactants, is nonflammable, is relatively inexpensive and is readily available in large amounts. Furthermore, carbon dioxide is a fluid which exhibits the possibility of being handled in the liquid state at relatively low temperatures and has the distinguishing feature of existing, in the supercritical state, at temperatures and pressures which are not very high.

Finally, under these operating conditions, carbon dioxide is nontoxic to human beings, animals and plants, which renders it entirely appropriate for the preparation of capsules which can be used in the fields relating to human, animal or plant health. Of course, other gaseous solvents known for behaving like carbon dioxide can be used.

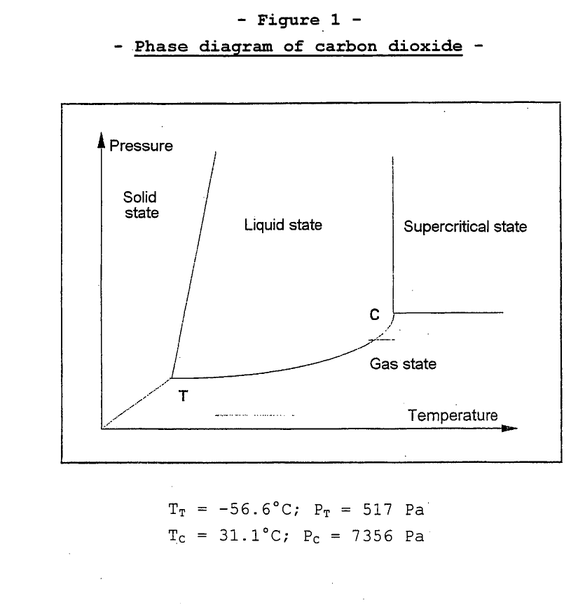

FIG. 1 shows the phase diagram of carbon dioxide. The point T, at the limit of the solid, liquid and gas states, is defined by its temperature T_{T} and its pressure P_{T}, respectively equal to −56.6° C. and 517 Pa. The point C, at the limit of the gas, liquid and supercritical states, is defined by its temperature T_{C} and its pressure P_{C}, respectively equal to 31.1° C. and 7356 Pa.

In its liquid and supercritical states, carbon dioxide can thus prove to be a suitable solvent of the organic phase during the interfacial polycondensation reaction in a dispersed medium. At the end of the reaction, it is sufficient to return to standard temperature and pressure conditions, under which conditions carbon dioxide is in the gas state, to easily remove this organic solvent. The obtained capsules will thus be devoid of organic solvent, generally responsible for their nonbiocompatibility.

However, the fact remains that the use of carbon dioxide in the liquid or supercritical state necessitates operating at relatively high pressures and at temperatures varying from approximately −60° C. up to approximately 200° C. The interfacial polycondensation reaction according to the invention will consequently be carried out in leaktight equipments which withstand such pressures and which is equipped with jackets which are both heating and cooling.

Furthermore, the density of liquid or supercritical carbon dioxide quite strongly varies with the temperature and the pressure. Variations in density result in variations in surface tension of the carbon dioxide which can disturb, indeed even upset, the equilibrium of the dispersion of the two organic and aqueous phases. It is therefore necessary to control the temperature and the pressure throughout the synthesis of the capsules and to keep them as constant as possible.

The interfacial polycondensation reaction will thus be carried out under the temperature and pressure conditions corresponding to those of the liquid or supercritical state of the normally gaseous solvent in which it is desired to carry out the reaction. Preferably, the dispersing will be carried out in a normally gaseous solvent, in the liquid state, preferably carbon dioxide in the liquid state. The strictly speaking polycondensation reaction can then be carried out either in a normally gaseous solvent, in the liquid state, preferably carbon dioxide in the liquid state, or, by modifying the temperature and/or the pressure, in a supercritical medium.

As has just been stated, the dispersing and the polycondensation reaction itself can be carried out at temperatures and pressures which vary within wide limits, provided that the normally gaseous solvent is in the desired state and within the limits of resistance of the equipment used. By way of example, the polycondensation reaction can be carried out at a temperature in the region of 60° C. and under a pressure of 15 MPa, when carbon dioxide is employed.

Carbon dioxide, in the liquid or supercritical state, is therefore a solvent suitable for the synthesis of capsules by interfacial polycondensation in a dispersed medium. As indicated above, the use of a surface-active agent is necessary to stabilize the dispersion before carrying out the interfacial polycondensation reaction. In particular, when the normally gaseous solvent used is carbon dioxide in the liquid state or in the supercritical state, it may prove to be necessary to employ amphiphilic surfactants, specific to water/liquid carbon dioxide or water/supercritical carbon dioxide.

Such surfactants are well known to a person skilled in the art and can advantageously be selected from compounds possessing polyfluorinated chains. The nature of the polyfluorinated surfactant is however not detrimental, provided the latter is soluble in liquid or supercritical carbon dioxide and exhibits an appreciable surfactant power. It will also preferably be selected for its biocompatibility and in particular its nontoxicity to the living environment.

A particularly advantageous example of a surfactant which is compatible with carbon dioxide is a compound possessing a fluorinated poly(propylene oxide) chain terminated by a carboxylic acid functional group. This compound is better known under the name Krytox® 157 FSL from DuPont. It can be used as is in its acid form or in the form of salts, for example sodium or ammonium salts, or in the form of a colloid, obtained, inter alia, with poly(1,4-butanediol toluene diisocyanate (PBTDI).

The process for the preparation of biocompatible capsules according to the present invention is preferably carried out using water as solvent of the lipophobic phase and carbon dioxide as solvent of the lipophilic phase. Of course, the monomers M₁ and M₂ must be selected so that they can react with one another to form a polycondensate and must be soluble, one in the lipophobic phase and the other in the lipophilic phase.

The monomers M₁ and M₂ are bifunctional, trifunctional or polyfunctional monomers, that is to say that they each comprise at least two reactive functional groups, possibly different but preferably identical, per molecule. Advantageously, the monomers M₁ and M₂ are bifunctional monomers, this bifunctionality ensuring the formation of polymers of high molecular weight which are particularly suitable for the formation of capsules in the interfacial system of the invention.

The presence of monofunctional monomers can reduce, indeed even halt, the growth of the polymer chains before they have reached the length suitable for the formation of the capsules. Conversely, poly-functionalized monomers will behave like crosslinking agents, thus resulting in a very rapid increase in the molecular mass of the polymer formed, the formation of a three-dimensional network and, very often, precipitation of the macromolecule.

Such monofunctional and polyfunctional monomers can, however, be used for the synthesis of the capsules of the present invention. In particular, polyfunctional monomers can advantageously be used as crosslinking agent in order to accelerate and/or to promote the formation of the polymer membrane.

Furthermore, in order to remain in accordance with the bio-compatible nature of the capsules set out here, it is desirable for the copolymer obtained by polycondensation of the monomers M₁ and M₂, and which forms the membrane of such capsules, to be biocompatible and non-toxic for the uses for which they are intended. This same biocompatibility should also be observed for the decomposition products of the polymer, which decomposition can, for example, occur by chemical decomposition or biochemical decomposition (metabolization).

Such biocompatible polymers exist and are commercially available today. Mention may be made, as examples, of Pellethane® (poly[ether-urethane]), Tecoflex® (poly[ether-urethane]), Biomer® (poly[ether-urethane-urea]) and Cardiothane® or Avcothane® (poly[ether-urethane] crosslinked with polydimethylsiloxane).

However, the process of the invention is not limited to the monomers which form biocompatible polymers, although the latter are recommended for the production of “clean” capsules, that is to say capsules which are biocompatible and nontoxic. Use will thus be made of monomers resulting, for example, in polymers selected from polyamides, polyesters, polyurethanes, polyureas and poly(ether-urethane-urea)s, in addition to their copolymers.

Of course, it should be understood that the term “monomer” employed in the present invention comprises not only monomers in the literal sense but also oligomers, telomers and others having characteristics similar to those of the monomers described above and resulting in constituent polymers suitable for the present invention of the membranes of the capsules. It is also possible to encompass using mixtures of monomers, oligomers, telomers and others or also replacing or adding a monomer, oligomer, telomer or other substance during the interfacial polycondensation reaction.

These constituent polymers of the membrane of the capsules can, for example, be obtained with the following pairs of monomers:

| “Aqueous” | | |
|---|---|---|
| monomers | “Organic” monomers | Polymers |
| Diol | Di(acid chloride) | Polyester |
| Diol | Diisocyanate | Polyurethane |
| Diol | Polyether functionalized | Poly(ether-urethane) |
| | with diisocyanate | |
| Diamine | Di(acid chloride) | Polyamide |
| Diamine | Diisocyanate | Polyurea |
| Diamine | Polyether functionalized | Poly(ether-urethane-urea) |
| | with diisocyanate | |

The “aqueous” monomers (that is to say, water-soluble monomers, i.e. monomers which are soluble in the lipophobic phase) which can advantageously be used in the context of the present invention are, for example, selected from alkanediols, such as 1,4-butanediol or 1,5-pentanediol, from poly(ethylene glycol oxide)s (PEGO) of various molecular masses, from alkanepolyols, for example alkanetriols, such as trimethylolpropane, and from di- or polyamines, for example 1,6-hexamethylenediamine, 1,2-ethylenediamine and tri(2-aminoethyl)amine.

Without implied limitation, the “organic” monomers (that is to say, fat-soluble monomers, i.e. monomers which are soluble in the organic phase or alternatively monomers which are soluble in carbon dioxide in the liquid and/or supercritical state) which can advantageously be used in the context of the present invention are, for example, selected from diisocyanates, for example methylenediphenyl isocyanate (MDI), 4,4′-dicyclohexylmethane diisocyanate (H₁₂MDI), toluene diisocyanate (TDI) or poly(1,4-butanediol toluene diisocyanate (PBTDI), and polyfunctional aliphatic polyisocyanates, for example Desmodur® N100, sold by Bayer. The “organic” monomers can also be di(acid chloride)s, such as terephthaloyl dichloride or sebacoyl dichloride.

The polycondensation reaction can also be carried out in the presence of one or more crosslinking agents advantageously selected from polyfunctional monomers, preferably trifunctional monomers, for example mesoyl trichloride.

Depending on the characteristics of the capsules which it is desired to prepare, it may be advantageous to carry out a polycondensation between one or more “organic” monomers and one or more “aqueous” monomers. The polycondensates thus obtained will be random or nonrandom copolymers of two, three, four or more monomers.

For the uses of the present invention, use will advantageously be made of an “aqueous” monomer of diol type and an “organic” monomer of diisocyanate type, so as to form capsules with a membrane which will be essentially composed of polyurethane.

The polycondensation reactions between the monomers M₁ and M₂ defined above can optionally be accelerated under the action of one or more catalysts. The nature of the catalyst to be employed depends on the nature of the monomers which have to react with one another and are fully known to a person skilled in the art who is a specialist in the synthesis of macromolecules.

By way of example, for a polycondensation between a diol and a diisocyanate, it may prove to be advantageous to accelerate the polycondensation reaction using a tin-based compound, such as Kosmos® 29 (K29) from Goldschmidt, or a tertiary diamine, such as DABCO (1,4-diazabicyclo[2.2.2]octane), sold by Aldrich, it being possible for these two catalysts to be used together. The tertiary diamine is preferred to the tin-based compound because of its better biocompatibility and its low toxicity, the amounts of catalysts employed, however, remaining very low.

Finally, the use of cosolvents, whether in the aqueous phase or in the organic phase (gaseous solvent, such as carbon dioxide), is not ruled out. This is because such cosolvents may be necessary to improve the solubility of the monomers in the water or in the solvent in the liquid or supercritical state.

The choice of such cosolvents should therefore be made according to the nature of the monomers involved in the interfacial polycondensation reaction and the optional surfactants and catalysts present in the reaction medium. Furthermore, these cosolvents should advantageously exhibit the feature of nontoxicity and better still of biocompatibility required for the uses defined in the present patent application.

The composite capsule thus obtained is composed of a polymer membrane resulting from the polycondensation of the monomers defined above, that is to say a polyamide, polyester, polyurethane, polyurea, poly(ether-urethane) or poly(ether-urethane-urea) polymer or alternatively a copolymer of these polymers, and of a core filled with the active substance or substances which will have been introduced beforehand into the dispersed phase.

The amount of encapsulated active substance will depend on the size of the capsules, which is generally monodisperse. Generally, the amount of active substance introduced into the dispersed phase will be between approximately 0.01% and approximately 99.9% by weight with respect to the total weight of the capsules, this being the case for capsules with a size of between approximately 0.01 μm and approximately 5 mm in diameter.

The limit of 0.01% indicates the lower limit below which the amount of active substance might not represent an effective amount for the use for which the capsule is intended. However, the encapsulation of active substances in amounts below this limit can be envisaged.

Above 99.9% of active substance with respect to the total weight of the capsule, the latter may no longer exhibit, for certain uses, a sufficiently high mechanical strength or alternatively a sufficiently sturdy membrane. Here again, capsules comprising more than 99.9% by weight of active substance with respect to the total weight of the capsule can be envisaged, however.

On conclusion of the polycondensation reaction, the capsules are recovered after evaporation of the gaseous solvent under standard temperature and pressure conditions (for example, carbon dioxide) and are washed with water. The capsules can subsequently be left in water or alternatively can be dried and/or dehydrated, indeed even lyophilized, in order to be stored.

The active substances which can be encapsulated can be of any kind and, for example, can be selected from therapeutic active principles for pharmaceutical or veterinary use, active principles for cosmetic uses, substances which are active from the agrochemical or plant-protection viewpoint, and the like.

There exists no specific limitation on the active substance which can be encapsulated according to the process of the present invention. Generally, the active substance is soluble in the dispersed phase, it being possible for this solubilization, if appropriate, to be facilitated in the presence of cosolvents as defined above. In addition, in some cases, the active substance can itself be the dispersed phase comprising the monomer.

The composite capsules obtained according to the process of the present invention are novel and consequently form an integral part of the invention. These capsules are characterized in that they have a membrane resulting from the interfacial polycondensation of at least two monomers and the solvent of which, used in the liquid and/or supercritical state, has been removed in the gas form under standard temperature and pressure conditions. Advantageously, the solvent used in the liquid and/or supercritical state removed in the gas form is the solvent of the lipophilic phase.

The fields of application of such biocompatible capsules, which make possible the delayed, sustained, prolonged and/or controlled release and/or the vectorization of the active principle or principles which they include, are very varied but relate in particular, because of their biocompatible nature, to the fields of human and animal health, plant protection and cosmetics, in general.

Thus, the biocompatible composite capsules of the invention can advantageously be used in the therapeutic, biomedical, pharmaceutical, veterinary, paramedical, cosmetological and plant-protection fields. By way of examples, and without implied limitation, the capsules can thus be used as vectorization agent for active principles and provide a rapid or slow, delayed, prolonged and/or controlled release of said active principles, depending on the nature of the membrane, the nature of the active substance or substances encapsulated, and the presence and the nature of various adjuvants, fillers, and the like, which can participate in the composition of said capsule.

More specifically, the capsules according to the invention can, for example, participate in the manufacture of pharmaceutical, veterinary, cosmetological or plant-protection products, such as tablets, hard gelatin capsules, powders, patches, gels, creams or ointments, but also in the manufacture of products for medical imaging (for example, contrast agent) or of textile products in the medical and/or paramedical field.

The present invention is described in more detail in the specific implementational examples which follow. These examples have only a purely illustrative nature and do not in the least have the aim of limiting the invention in any way whatsoever.

### EXAMPLE 1

### Synthesis of Minicapsules with a Polyurethane Membrane which are Obtained by Interfacial Polycondensation in a Conventional Organic Solvent Medium

The reaction begins by the preparation of the dispersing phase, obtained by dissolving the surfactant (Hypermer® B261) in 1 L of organic solvent (cyclohexane or toluene) in order to obtain a concentration of 1 g·L⁻¹. A portion (450 mL) of this solution is introduced into a reactor equipped with a bulb condenser. The solution is subsequently degassed for 30 min by bubbling with nitrogen, before increasing the temperature to 65° C., after having closed the reactor.

The dispersed phase, comprising the water-soluble monomers, is prepared by dissolving the diol, the crosslinking agent (trimethylolpropane) and, if appropriate, the catalyst (DABCO) in 50 mL of water.

The dispersed phase is then dispersed for 5 minutes in the organic solution by introducing it rapidly into the reactor, where stirring was begun beforehand at 800 rpm, a speed appropriate for producing minicapsules under these conditions.

During this time, an organic solution, comprising the fat-soluble monomers, is prepared from 50 mL of dispersing phase, in which the diisocyanate, the crosslinking agent (Desmodur® N100) and, if appropriate, the catalyst Kosmos® 29 (K29) are dissolved.

Once the dispersion has been produced, the stirring speed is lowered to 200 rpm, an optimum speed which makes possible good mixing of the reaction medium while preventing damage to the capsules. The organic solution comprising the fat-soluble monomers is then introduced slowly into the reactor using a syringe fitted to a syringe driver adjusted to make possible a flow rate for addition of approximately 2.5 mL/min.

The reaction mixture is then left for 4 hours to allow the polycondensation reaction to occur.

At the end of the reaction, 450 mL of dispersing phase are again introduced into the reactor in order to dilute the unreacted fat-soluble reactants, thus halting the polycondensation reaction. In fact, the reaction is not truly halted but greatly slowed down.

The reactor is then bled and the dispersion is filtered through a filter paper. The minicapsules are then rinsed several times in an organic solution of dispersing phase (prepared at the beginning of handling), before being gently dried on absorbent paper in order to remove as much as possible of the organic solvent.

Finally, the minicapsules are washed in aqueous solutions comprising a surfactant which makes it possible to prevent the agglomeration of the capsules and to remove the final traces of organic solvent absorbed at the surface of the capsules. These aqueous washing solutions are prepared from a 1 L mother solution comprising 0.5% by volume of surfactant (Tween® 20). The washing operations are then carried out with decreasing concentrations of surfactant, until the smell of organic solvent has faded away. The mini-capsules are then stored in water.

Capsules with a polyurethane membrane which enclose water and which have a mean diameter of between 100 μm and 300 μm are thus obtained. The entire phase is encapsulated with a degree of encapsulation in the region of 100%.

### EXAMPLE 2

### Synthesis of Minicapsules by Interfacial Polycondensation in a Liquid Carbon Dioxide Medium

The reactor used in this example (volume=100 mL) is equipped with a pressure-equalizing dropping funnel with a volume of 400 mL.

1.85 mmol of methylenediphenyl isocyanate, 0.55 mmol of Desmodur® N 100 and 500 mg of surfactant Krytox® 157 FSL, in the ammonium salt form, are introduced into the reactor. The reactor is then hermetically sealed.

The aqueous solution is subsequently prepared from 5 mL of water, in which 1.57 mmol of 1,5-pentanediol, 0.47 mmol of trimethylolpropane and 23 mg of 1,4-diazabicyclo[2.2.2]octane (DABCO) are dissolved. This solution is introduced into the dropping funnel.

The combined apparatus (reactor+pressure-equalizing dropping funnel) is then purged twice to 3 MPa of carbon dioxide (CO₂) pressure before introducing 140 g of CO₂ (or else 70 g of CO₂ into the reactor only when the dropping funnel is isolated).

Stirring is begun at 200 rpm and the heating is brought to a temperature of 30° C., which facilitates the dissolution of the reactants in the CO₂. When the temperature is reached, stirring is increased up to 800 rpm and the dispersed phase present in the pressure-equalizing dropping funnel is rapidly introduced.

After reacting for 5 minutes, stirring is reduced to 200 rev/min and then the reaction mixture is left overnight (approximately 16 hours) in order to make sure that all the monomer has been consumed.

At the end of the reaction, stirring and heating are halted and then, when the reaction medium has returned to ambient temperature, the degassing phase is begun: a pressure-reducing valve, connected to the reactor, provides a constant pressure of 100 kPa at the outlet of the latter, which is connected to a flow meter. The flow rate of the gas at the outlet is set at 20 mL/min.

At the end of degassing, the rapid bleed valve is opened in order to make sure that the assembly is no longer under pressure. The reactor is then opened and the reaction products are then recovered.

Composite capsules formed of a core of water and of a polyurethane membrane are obtained, the capsules having a mean size (diameter) of between 100 μm and 300 μm.

### EXAMPLE 3

### Synthesis of Minicapsules by Interfacial Polycondensation in a Supercritical Carbon Dioxide Medium

The method of preparation is identical to that set out in Example 2, apart from the fact that, as the dispersion is produced in liquid CO₂, that is to say in CO₂ at a temperature of less than 30° C., the temperature is subsequently brought to the desired value in order for the CO₂ to be in the supercritical phase, that is to say approximately 60° C.

The capsules thus obtained are formed of a core of water and of a polyurethane membrane. Their mean diameter is between 100 μm and 300 μm.

1-29. (canceled)

30. A process for the preparation of composite capsules by interfacial polycondensation in a dispersed medium of at least two different monomers M₁ and M₂, characterized in that said polycondensation is carried out in a gas in the liquid and/or supercritical state.

31. The process as claimed in claim 30, characterized in that it comprises the following steps:
a) a solution of at least one monomer M₁ in a solvent S₁ comprising the substance to be encapsulated is added, with stirring, to a solution comprising a solvent S₂, in order to obtain a stabilized dispersion, S₁ or S₂ being a gas in the liquid and/or supercritical state;
b) a solution comprising at least one monomer M₂ present in the minimum amount of S₂ is added to this dispersion;
c) the polymerization is carried out by polycondensation at the interface of the two liquids;
d) the gas present in the reaction medium is removed; and
e) the capsules are extracted from the reaction medium.

32. The process as claimed in claim 30, in which S₁ is the solvent of the lipophobic phase.

33. The process as claimed in claim 30, in which S₁ is the solvent of the lipophilic phase.

34. The process as claimed in claim 30, in which S₁ is water and S₂ is the gas in the liquid and/or supercritical state.

35. The process as claimed in claim 30, characterized in that the gas is selected from air, oxygen, nitrogen, nitrous oxide, carbon dioxide, the rare gases, halogenated or nonhalogenated hydrocarbons, and their mixtures in all proportions.

36. The process as claimed in claim 30, characterized in that the gas is nontoxic to man, animals and/or plants.

37. The process as claimed in claim 30, characterized in that the gas is carbon dioxide.

38. The process as claimed in claim 30, in which the dispersion comprises a surface-active agent.

39. The process as claimed in claim 30, characterized in that the dispersion comprises a surfactant which makes it possible to avoid the dispersed droplets in the course of growing from agglomerating with one another.

40. The process as claimed in claim 38, in which the surfactant is a compound possessing polyfluorinated chains.

41. The process as claimed in claim 40, in which the surfactant is a compound possessing a fluorinated poly(propylene oxide) chain terminated by a carboxylic acid functional group.

42. The process as claimed in claim 30, characterized in that the monomers M₁ and M₂ are bifunctional, trifunctional or polyfunctional monomers.

43. The process as claimed in claim 42, characterized in that the monomers are selected, for the monomers of the lipophobic phase, from diamines and diols and, for the monomers of the lipophilic phase, from di(acid chloride)s, diisocyanates and polyethers functionalized with diisocyanate.

44. The process as claimed in claim 43, characterized in that the lipophobic monomers are selected from alkanediols, such as 1,4-butanediol or 1,5-pentanediol, poly(ethylene glycol oxide)s (PEGO) of various molecular masses and alkanepolyols, for example alkanetriols, and from di- or polyamines, for example 1,6-hexamethylenediamine, 1,2-ethylenediamine and tri(2-aminoethyl)amine.

45. The process as claimed in claim 44, characterized in that the monomers of the lipophilic phase are selected from diisocyanates, for example methylenediphenyl isocyanate (MDI), 4,4′-dicyclohexylmethane diisocyanate (H₁₂MDI), toluene diisocyanate (TDI) or poly(1,4-butanediol toluene diisocyanate) (PBTDI), from polyfunctional aliphatic polyisocyanates and from di(acid chloride)s, such as terephthaloyl dichloride or sebacoyl dichloride.

46. The process as claimed in claim 30, characterized in that the monomers are selected from diols as monomers of the lipophobic phase and from di(acid chloride)s as monomers of the lipophilic phase.

47. The process as claimed in claim 30, characterized in that the monomers are selected from diols as monomers of the lipophobic phase and from diisocyanates as monomers of the lipophilic phase.

48. The process as claimed in claim 30, characterized in that the monomers are selected from diamines as monomers of the lipophobic phase and from di(acid chloride)s as monomers of the lipophilic phase.

49. The process as claimed in claim 30, characterized in that the monomers are selected from diamines as monomers of the lipophobic phase and from diisocyanates as monomers of the lipophilic phase.

50. The process as claimed in claim 30, characterized in that the polycondensation reaction is carried out in the presence of a polycondensation catalyst.

51. A composite capsule, characterized in that it has a membrane resulting from the interfacial polycondensation of at least two monomers and the solvent of which, used in the liquid and/or supercritical state, has been removed in the gas form under standard temperature and pressure conditions.

52. The composite capsule as claimed in claim 51, characterized in that it is composed of a core filled with one or more active substances and of a membrane made of polyamide, polyester, polyurethane, polyurea, poly(ether-urethane) or poly(ether-urethane-urea) polymer or copolymer of these polymers.

53. The composite capsule as claimed in claim 51, characterized in that its mean external diameter is between approximately 0.01 μm and approximately 500 μm, preferably between approximately 0.05 μm and approximately 300 μm.

54. The composite capsule as claimed in claim 53, characterized in that its mean external diameter is between approximately 0.01 μm and approximately 500 μm, preferably between approximately 0.05 μm and approximately 200 μm.

55. The composite capsule as claimed in claim 51, characterized in that it comprises between 0.01% and 99.9% by weight of active material(s).

56. The use of the composite capsule as claimed in claim 51 for the delayed, sustained, prolonged and/or controlled release and/or the vectorization of the active principle or principles which it encloses.

57. The use as claimed in claim 56 for the manufacture of pharmaceutical, veterinary, plant-protection or cosmetic products.

58. A pharmaceutical, veterinary, plant-protection or cosmetic product, comprising the composite capsule as claimed in claim 51.
